(19) United States
(12) Patent Application Publication
(10) Pub. No.: US 2002/0123791 A1
(43) Pub. Date: Sep. 5, 2002

(54) Stent design with increased vessel coverage

(76) Inventor: William J. Harrison, Temecula, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 09/750,474

(22) Filed: Dec. 28, 2000

(51) Int. Cl. A61F 2/06

(52) U.S. Cl. 623/1.15

(57) ABSTRACT

A stent with an increased vessel coverage includes a plurality of radially expandable cylindrical elements generally arranged on a common longitudinal stent axis and interconnected by one or more interconnecting members placed so that the stent remains flexible in a longitudinal direction. Each cylindrical element is formed in a generally serpentine wave pattern having alternating valley and peak portions which is capable of nesting when crimped or placed in a compressed condition. The valley portions and peak portions may be V-shaped and W-shaped elements which have different longitudinal lengths which permit the nesting of the cylindrical element. The stent can be made to be expandable by an external force, such as a balloon expandable dilatation catheter, or can be self-expanding when made from a material which is self-expanding.

### BACKGROUND OF THE INVENTION

The present invention relates to expandable endoprosthesis devices, generally called stents, which are adapted to be implanted into a patient's body lumen, such as carotid arteries, coronary arteries, peripheral arteries, veins, or other vessels to maintain the patency of the lumen. More particularly, the invention relates to the design and configuration of the stent struts which provide increased vessel coverage to help minimize the disturbance to the blood flow in the vessel, to provide enhanced scaffolding of the wall of the body lumen, and to minimize the trauma caused by the stent to the body lumen in which it is implanted.

Stents are frequently used in the treatment of atherosclerotic stenosis in blood vessels especially in conjunction with percutaneous translumenal angioplasty (PTA) or percutaneous translumenal coronary angioplasty (PTCA) procedures, with the intent to reduce the likelihood of restenosis of a vessel. Stents are also used to support a body lumen, tack-up a flap or dissection in a vessel, or in general, where the lumen is weak to add support. Stents are generally cylindrically shaped devices which function to hold open and sometimes expand a segment of a blood vessel or other arterial lumen, such as a coronary artery. Stents are usually delivered in a compressed condition to the target site and then deployed at that location into an expanded condition to support the vessel and help maintain it in an open position. They are particularly suitable for use in supporting and holding back a dissected arterial lining which can occlude the fluid passageway there through.

Stents or expandable grafts are implanted in a variety of body lumens in an effort to maintain their patency and are especially well-suited for the treatment of atherosclerotic stenosis in blood vessels. Intracoronary stents have become a standard adjunct to percutaneous coronary angioplasty in the treatment of arterial atherosclerotic disease. Although commercial stents vary in design and materials, they share similar structural features. Most stents in clinical use today are metallic and are either self-expanding or are expanded by the force of an expandable member, such as an angioplasty dilatation balloon. These devices are typically implanted via a delivery catheter which is inserted at an easily accessible location on the patient and then advanced through the patient's vasculature to the deployment site. The stent is initially maintained in a radially compressed or collapsed state to enable it to be maneuvered through the lumen and into the stenosis. Once in position, the stent is deployed which, depending upon its construction, is achieved either automatically by the removal of a restraint, or actively by the inflation of a balloon about which the stent is carried on the delivery catheter.

The stent must be able to simultaneously satisfy a number of mechanical requirements. First and foremost, the stent must be capable of withstanding the structural loads that are imposed thereon as it supports the lumen wall. In addition to having adequate radial strength or more accurately, hoop strength, the stent should nonetheless be longitudinally flexible to allow it to be maneuvered through a tortuous vascular path and to enable it to conform to a deployment site that may not be linear or may be subject to flexure. The material of which the stent is constructed must allow the stent to undergo expansion, which typically requires substantial deformation of localized portions of the stent's structure. Once expanded, the stent must maintain its size and shape throughout its service life to properly support the vessel wall despite the various forces that may come to bear upon it, including the cyclic loading induced by the pulsatile character of arterial blood flow. Finally, the stent must be biocompatible so as not to trigger any adverse vascular responses. A variety of devices are known in the art for use as stents and have included coiled wires in a variety of patterns that are expanded after being placed intralumenally on a balloon catheter, helically wound coiled springs manufactured from an expandable heat sensitive metal, and self-expanding stents inserted into a compressed state for deployment into a body lumen. One of the difficulties encountered in diagnosing prior art stents involve maintaining the radial rigidity needed to hold open a body lumen while at the same time maintaining the longitudinal flexibility of the stent to facilitate its delivery and accommodate the often tortuous path of the body lumen.

As mentioned above, prior art stents typically fall into two general categories of construction. The first type of stent is mechanically-expandable upon application of a controlled force, often through the inflation of the balloon portion of a dilatation catheter which, upon inflation of the balloon or other expansion means, expands the compressed stent to a larger diameter to be left in place within the artery at the target site. The second type of stent is a self-expanding stent formed from, for example, shape memory metals or super-elastic nickel-titanum (NiTi) alloys, which will automatically expand from a compressed state when the stent is advanced out of the distal end of the delivery catheter into the body lumen. Many of these stents manufactured from expandable heat sensitive materials allow for phase transformations of the material to occur, resulting in the expansion and contraction of the stent.

Details of prior art mechanically-expandable stents can be found in U.S. Pat. No. 3,868,956 (Alfidi et al.); U.S. Pat. No. 4,512,1338 (Balko et al.); U.S. Pat. No. 4,553,545 (Maass, et al.); U.S. Pat. No. 4,733,665 (Palmaz); U.S. Pat. No. 4,762,128 (Rosenbluth); U.S. Pat. No. 4,800,882 (Gianturco); U.S. Pat. No. 5,514,154 (Lau, et al.); U.S. Pat. No. 5,421,955 (Lau et al.); U.S. Pat. No. 5,603,721 (Lau et al.); U.S. Pat. No. 4,655,772 (Wallsten); U.S. Pat. No. 4,739,762 (Palmaz); and U.S. Pat. No. 5,569,295 (Lam). Further details of prior art self-expanding stents can be found in U.S. Pat. No. 4,580,568 (Gianturco); and U.S. Pat. No. 4,830,003 (Wolff, et al.).

Further details of prior art self-expanding stents can be found in U.S. Pat. No. 4,580,568 (Gianturco); and U.S. Pat. No. 4,830,003 (Wolff, et al.).

Mechanically-expandable stents are delivered to the target site by delivery systems which often use balloon catheters as the means for delivering and expanding the stent in the target area. One such stent delivery system is disclosed in U.S. Pat. No 5,158,548 to Lau et al. Such a stent delivery system has an expandable stent in a contracted condition placed on an expandable member, such as an inflatable balloon, disposed on the distal portion of an elongated catheter body. A guide wire extends through an inner lumen within the elongated catheter body and out its distal end. A tubular protective sheath is secured by its distal end to the portion of the guide wire which extends out of the distal end of the catheter body and fits over the stent mounted on the expandable member on the distal end of the catheter body.

Some prior art stent delivery systems for implanting self-expanding stents include an inner lumen upon which the compressed or collapsed stent is mounted and an outer restraining sheath which is initially placed over the compressed stent prior to deployment. When the stent is to be deployed in the body vessel, the outer sheath is moved in relation to the inner lumen to “uncover” the compressed stent, allowing the stent to move to its expanded condition into the target area.

Despite the widespread use of stents, in-stent restenosis remains a major clinical problem; however, restenosis does not develop in all patients undergoing coronary angioplasty and stent implantation. The mechanism of restenosis after stent implantation is principally neointimal hyperplasia, as stents resist negative arterial remodeling. Relative to PTCA alone, stents improve the outcome by minimizing vessel recoil, reducing plaque prolapse, and affecting long term remodeling.

While there are numerous benefits associated with stent implantation, it is also well-known that stent struts can alter the flow of blood. Immediately upstream and downstream of the struts, the flow can be disturbed, with flow reversals and eddies. Often, the flow is disturbed by tissue prolapse which results when there are openings within the stent strut boundaries. If there is too much open area, some tissue from the body lumen can extend through openings between the stent struts and enter into the stent lumen which results in disturbed blood flow. For this reason, the stent structure must provide sufficient scaffolding within the body lumen to help prevent tissue prolapse. This disturbance in normal blood flow can result in abnormal cell proliferation which causes the lumen to narrow and potentially sets up the stage for further atherosclerotic disease. Proper vessel scaffolding can help to decrease blood flow disturbances within the inner surface of the stent while promoting proper growth of smooth muscle cell proliferation to reduce the chances of restenosis. However, excessive surface coverage by the stent can still result in greater thrombogenicity, along with the loss of flexibility of the stent. Therefore, the stent design and the amount of surface metal coverage can affect not only the physical properties of the stent, but also the degree of vessel wall injury, along with the quality of the neointima formed after implantation of the stent.

What has been needed, and heretofore unavailable, in the art of stent design which minimizes neointimal growth and reduces the disturbance of the blood flow within the vessel while providing enhanced surface coverage of the stent struts to the lumenal wall. Moreover, the stent design should reduce the injury and inflammation of the vessel wall. Additionally, the expanded stent should have sufficient structural strength (hoop strength) to hold the body lumen open once expanded. Such a stent should also have sufficient radiopaque properties to permit it to be sufficiently visualized on external monitoring equipment, such as a fluoroscope, to allow the physician to properly position the stent in the target location and be collapsible/crimpable to attain a low profile device. The present invention satisfies these and other needs.

### SUMMARY OF THE INVENTION

Briefly, and in general terms, the present invention is directed to the design and configuration of stents that increase the amount of lumenal surface supported by the stent. The enhanced surface coverage provided by the present invention helps to minimize the disturbance of blood flow in the vessel along with the trauma caused by the stent to the vessel wall in which it is implanted. The stent design of the present invention helps prevent tissue prolapse from extending between the struts of the stent, when implanted, to create a smoother inner surface that contacts the blood flow, thus reducing the turbulence of the blood flow as it passes through the stent. Additionally, such a configuration should help reduce the disturbance to the endothelium created by changes in fluid shear stress, and minimizes trauma to the vessel wall, leading to a decrease in neointimal hyperplasia.

In all embodiments, the stents of the present invention have sufficient longitudinal flexibility along their longitudinal axis to facilitate delivery through tortuous body lumens, yet remain stable when expanded radially to maintain the patency of a body lumen such as an artery or other vessel, when implanted therein. The present invention in particular relates to unique patterns which permit greater longitudinal flexibility and sufficient radial-expansibility and strength to hold open the desired body lumens. The present invention also allows for closer placement of adjacent struts, referred to as nesting, which creates a stent design that can be crimped/collapsed to a low profile for delivery purposes. A low profile allows the stent to be placed in smaller, more distal vessels, such as those encountered in the coronary arteries.

The stents of the present invention include a plurality of adjacent cylindrical elements (often referred to as “rings”) which are generally expandable in the radial direction and arranged in alignment along a longitudinal stent axis. The cylindrical elements are formed in a variety of serpentine wave patterns transverse to the longitudinal axis and contain a plurality of alternating peaks and valleys. At least one interconnecting member extends between adjacent cylindrical elements and connects them to one another. These interconnecting members insure a minimal longitudinal contraction during radial expansion of the stent in the body vessel. The serpentine patterns have varying degrees of curvature in the regions of peaks and valleys and are adapted so that radial expansion of the cylindrical elements are generally uniform around their circumferences during expansion of the stent, whether from balloon expansion or self-expansion, from a contracted condition to the expanded condition.

In one aspect of the present invention, each cylindrical element of the stent includes six peak regions (often referred to as “crowns”) and six valley regions, with three interconnecting rings adjacent cylindrical elements. Each cylindrical element or ring is made from V-shaped, W-shaped or inverted V-shaped and W-shaped portions. The overall profile of the stent in its unexpanded or contracted condition (sometimes referred to as the “crimp profile”) can be reduced by decreasing the length of the W-shaped portions that are adjacent to inverted V-shaped portions to allow increased nesting during crimping or collapse of the stent. The resulting stent produces a six crown, three-cell pattern which has sufficient coverage for vessel scaffolding and maintains excellent flexibility to reach distal lesions, while still possessing sufficient radial strength to hold the target vessel open. The stent design also utilizes slightly smaller radii for the V-shaped and W-shaped portions, compared to some commercially available stents, to reduce the crimp size.

Preferably, the number and location of the interconnecting members can be varied in order to develop the desired longitudinal flexibility in the stent structure both in the unexpanded as well as the expanded state. The use of fewer interconnecting members usually results in a more flexible design since this “frees up” more of the highly flexible V-shaped peaks. Thus, flexibility is derived mainly from the rings while the number and location of the interconnecting members influences the flexibility by constraining or “freeing up” the V-shaped members. Generally, the greater the longitudinal flexibility of the stents, the easier and the more safely they can be delivered to the implantation site, especially where the implantation site is on a curved section of a body lumen, such as a coronary artery or peripheral blood vessel, and especially in saphenous veins and larger vessels. However, if increased vessel scaffolding is still desired, the number of interconnecting members can be increased as needed.

In another aspect of the present invention, four interconnecting members, rather than three, are utilized to connect adjacent cylindrical elements. In this particular stent design, the interconnecting members connect adjacent rings from peak to peak and valley to valley to increase the amount of surface area of the lumenal wall which is supported by the struts of the stent. In many current stent designs, interconnecting members usually are placed to connect only valley portions of one ring to valley portions of an adjacent ring or peak portion to peak portion. The present stent design which connects both peaks to peaks and valleys to valleys of adjacent cylindrical rings increases the amount of stent coverage in the body lumen while still providing longitudinal flexibility to maneuver through some tortuous anatomy.

In yet another aspect of the present invention, the stent design includes eight peak regions and eight crown regions per ring with four interconnecting members connecting adjacent rings. As a result, shorter strut arms can be utilized since a change in circumference is taken up by more V-shaped, W-shaped and inverted V-shape portions for each cylindrical ring. As a result, there are more rings for the same stent length than is utilized in conjunction with the other designs disclosed herein. The supported surface area in the body lumen can then be greatly enhanced by utilizing shorter rings and additional interconnecting members.

The resulting stent structures are a series of radially expandable cylindrical elements that are spaced longitunally close enough so that small dissections in the wall of a body lumen may be pressed back into position against the lumenal wall, yet does not compromise the longitudinal flexibility of the stent both when negotiating through the body lumens in their unexpanded state and when expanded into position. Each of the individual cylindrical elements may rotate slightly relative to their adjacent cylindrical elements without significant deformation, cumulatively providing stents which are flexible along their length and about their longitudinal axis, but which still are very stable in their radial direction in order to resist collapse after expansion.

The stents of the present invention can be readily delivered to the desired target location by mounting it on an expandable member, such as a balloon, of a delivery catheter and passing the catheter-stent assembly lumen to the target area. A variety of means for securing a stent to the extendible member of the catheter for delivery to the desired location are available. For example, the stent can be crimped or compressed onto the unexpanded balloon. The present design is particularly suitable for crimping since the nesting of the V-shaped and W-shaped portions of the cylindrical rings process a low profile suitable for crossing tight or distal lesions. Other means to secure the stent to the balloon included providing ridges or collars on the inflatable member to restrain lateral movement, using bioabsorbable temporary adhesives, or adding a retractable sheath to cover the stent during delivery through a body lumen. When a stent of the present invention is made from a self-expanding material, such as nickel titanium alloy, a suitable stent delivery assembly which includes a retractable sheath, or other means to hold the stent in its unexpanded condition prior to deployment, can be utilized.

These and other features and advantages of the present invention will become more apparent from the following detailed description of the invention, when taken in conjunction with the accompanying exemplary drawings.

### BRIEF DESCRIPTION OF THE DRAWINGS

FIG. 1 is an elevational view, partially in section, depicting a stent embodying features of the present invention mounted on a delivery catheter disposed within a vessel.

FIG. 2 is an elevational view, partially in section, similar to that shown in FIG. 1, wherein the stent is expanded within a vessel, pressing the lining against the vessel wall.

FIG. 3 is an elevational view, partially in section, showing the expanded stent within the vessel after withdrawal of the delivery catheter.

FIG. 4 is a plan view of one embodiment of a flattened stent of the present invention, which illustrates the serpentine pattern including peaks and valleys which form the cylindrical elements of the stent and permit the stent to achieve a small crimp profile, yet is expandable to a larger diameter to maintain the patency of a small vessel.

FIG. 5 is an enlarged partial view of the stent of FIG. 4 depicting the serpentine pattern along with the peaks and valleys which form one embodiment of a cylindrical element made in accordance with the present invention.

FIG. 5A is an enlarged partial view of the cylindrical element of FIG. 5 showing the nesting of the V-shaped portion and W-shaped portion when the stent is crimped or collapsed.

FIG. 6 is a plan view of another embodiment of a flattened stent of the present invention, which illustrates the serpentine pattern along with the peaks and valleys which form the cylindrical elements of the stent.

FIG. 7 is an enlarged partial view of the stent of FIG. 6 depicting the serpentine pattern along with the peaks and valleys which form another embodiment of a cylindrical element made in accordance with the present invention.

FIG. 8 is a plan view of another embodiment of a flattened stent of the present invention, which illustrates the serpentine pattern along with the peaks and valleys which form the cylindrical elements of the stent.

FIG. 9 is an enlarged partial view of the stent of FIG. 8 depicting the serpentine pattern along with the peaks and valleys which form another embodiment of a cylindrical element made in accordance with the present invention.

### DETAILED DESCRIPTION OF THE PREFERRED EMBODIMENTS

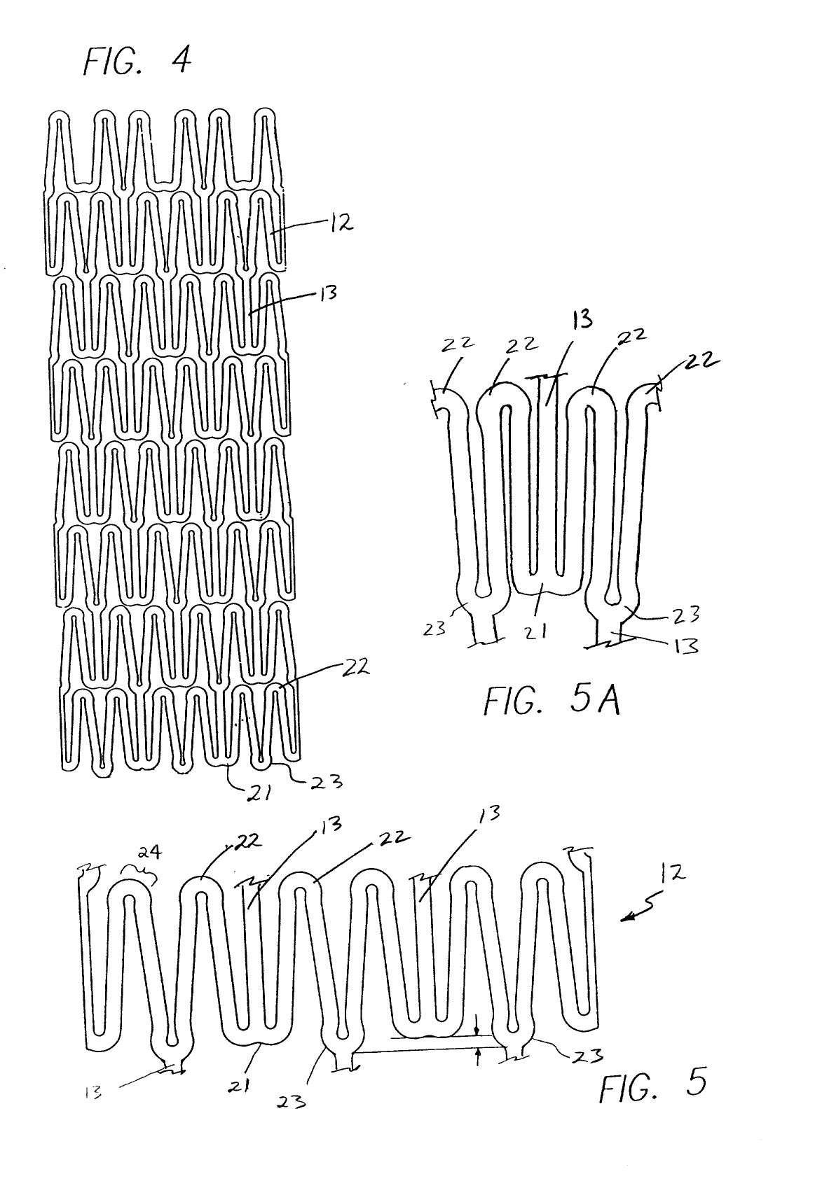

Turning now to the drawings in which reference numbers represent like or corresponding elements in the drawings, FIG. 1 illustrates an exemplary embodiment of stent 10 incorporating features of the present invention, which stent is mounted onto delivery catheter 11. FIG. 4 is a plan view of this embodiment of the stent 10 with the structure flattened out into two dimensions to facilitate explanation. Stent 10 generally comprises a plurality of radially expandable cylindrical elements or rings 12 disposed generally coaxially and interconnected by interconnecting members 13 disposed between adjacent cylindrical elements 12. The delivery catheter 11 has an expandable portion or balloon 14 for expanding stent 10 within artery 15 or other vessel. The artery 15, as shown in FIG. 1, has a dissected or detached lining 16 which has occluded a portion of the arterial passageway.

The delivery catheter 11 onto which stent 10 is mounted is essentially the same as a conventional balloon dilatation catheter for angioplasty procedures. The balloon 14 may be formed of suitable materials such as polyethylene, polyethylene terephthalate, polyvinyl chloride, nylon and, ionomers such as Surlyn® manufactured by the Polymer Products Division of the Du Pont Company. Other polymers may also be used.

In order for stent 10 to remain in place on balloon 14 during delivery to the site of the damage within artery 15, stent 10 is compressed or crimped onto balloon 14. Alternatively, retractable protective delivery sleeve (not shown) may be provided to ensure that stent 10 stays in place on balloon 14 of delivery catheter 11 and to prevent abrasion of the body lumen by the open surface of stent 10 during delivery to the desired arterial location. Other means for securing stent 10 onto balloon 14 also may be used, such as providing collars or ridges on the ends of the working portion, i.e., the cylindrical portion, of balloon 14. Each radially expandable cylindrical element 12 of stent 10 may be substantially independently expanded. Therefore, balloon 14 may be provided with an inflated shape other than cylindrical, e.g., tapered, to facilitate implantation of stent 10 in a variety of body lumen shapes. When the stent 10 is made from a self-expanding material such as Nitinol, a suitable delivery device with retractable sleeve may be used to deploy the stent.

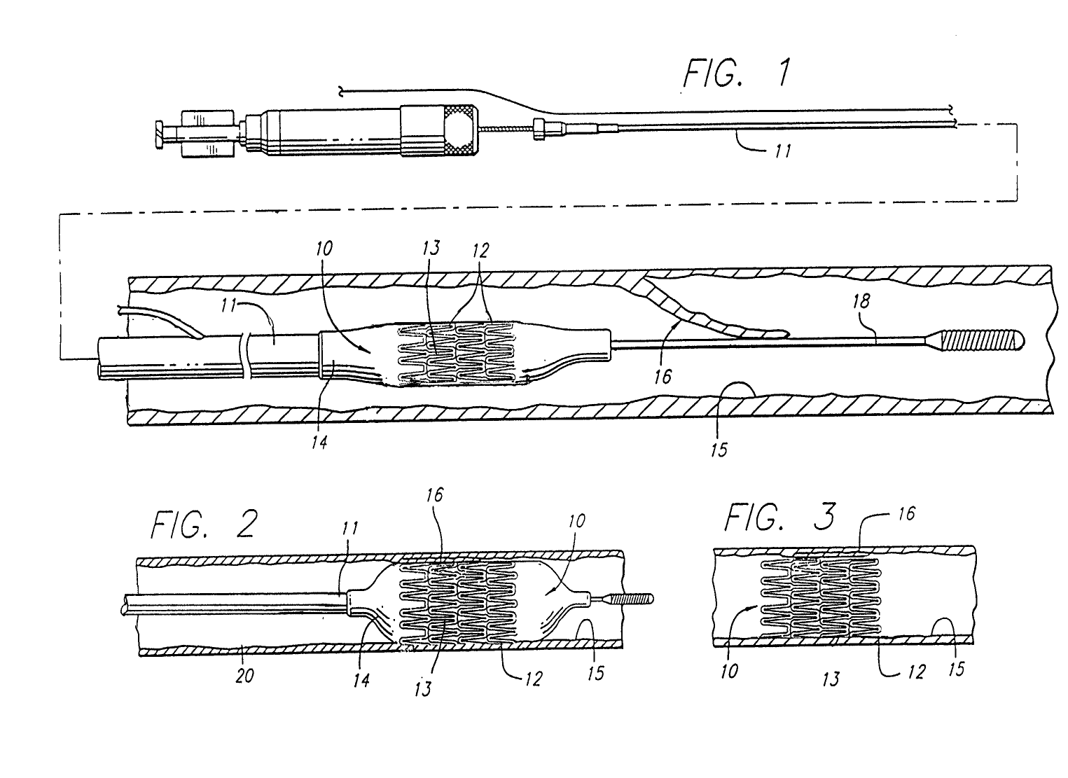

The delivery of stent 10 may be accomplished in the following manner. Stent 10 is first mounted onto inflatable balloon 14 on the distal extremity of delivery catheter 11. Stent 10 may be crimped down onto balloon 14 to obtain a low profile. The catheter-stent assembly can be introduced within the patient's vasculature in a conventional Seldinger technique through a guiding catheter (not shown). Guidewire 18 is disposed through the damaged arterial section with the detached or dissected lining 16. The catheter-stent assembly is then advanced over guide wire 18 within artery 15 until stent 10 is directly under detached lining 16. Balloon 14 of catheter 11 is inflated or expanded, thus expanding stent 10 against the inside of artery 15, which is illustrated in FIG. 2. While not shown in the drawing, artery 15 is preferably expanded slightly by the expansion of stent 10 to seat or otherwise embed stent 10 to prevent movement. Indeed, in some circumstances during the treatment of stenotic portions of an artery, the artery may have to be expanded considerably in order to facilitate passage of blood or other fluid there through. It should be apparent to those skilled in the art that this is just one manner of delivering a stent to an area of treatment. Those skilled in the art will appreciate that other techniques can be utilized in accordance with the present invention as well.

While FIGS. 1-3 depict a vessel having detached lining 16, stent 10 can be used for purposes other than repairing the lining. Those other purposes include, for example, supporting the vessel, reducing the likelihood of restenosis, or assisting in the attachment of a vascular graft (not shown) when repairing an aortic abdominal aneurysm. Additionally, as mentioned before, the present invention can be utilized in any number of different body lumens in the patient's vasculature, including the carotid arteries, coronary arteries, peripheral arteries, veins and other vessels to maintain the patency of the lumen.

In general, stent 10 serves to hold open artery 15 after catheter 11 is withdrawn, as illustrated in FIG. 3. Due to the formation of stent 10, the undulating component of the cylindrical elements of stent 10 is relatively flat in a transverse cross-section so that when stent 10 is expanded, cylindrical elements 12 are pressed into the wall of artery 15 and as a result do not interfere with the blood flow through artery 15. Cylindrical elements 12 of stent 10 that are pressed into the wall of artery 15 will eventually be covered with endothelial cell growth that further minimizes blood flow turbulence. The close spacing of the struts which form the cylindrical elements helps prevent tissue prolapse that can cause disruptive blood flow and abnormal cell proliferation that can cause lumenal narrowing. The serpentine pattern of cylindrical elements 12 provide good tacking characteristics to prevent stent movement within the artery. Furthermore, the closely spaced cylindrical elements 12 at regular intervals provide uniform support for the wall of artery 15, and consequently are well adapted to tack up and hold in place small flaps or dissections in the wall of artery 15 as illustrated in FIGS. 2 and 3.

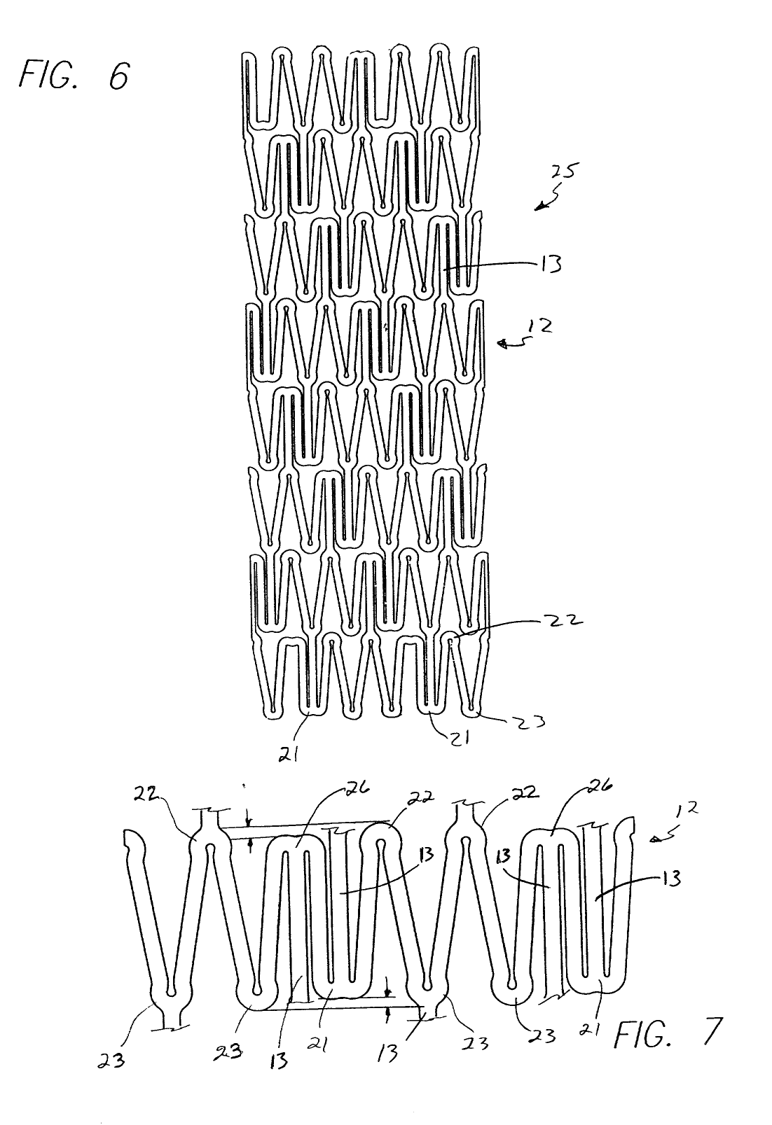

The stresses involved during expansion from a low profile to an expanded profile are generally evenly distributed among the various peaks and valleys of stent 10. Referring to FIGS. 4 and 5, one embodiment of the present invention as depicted in FIGS. 1-3 is shown wherein each expanded cylindrical element 12 embodies a serpentine pattern having a plurality of peaks and valleys that aid in the even distribution of expansion forces. In this embodiment, interconnecting members 13 serve to connect adjacent valleys of each adjacent cylindrical element 12 as described above. The various peaks and valleys generally have V, W and inverted V-shapes, in a repeating pattern to form each cylindrical element 12. It should be appreciated that the cylindrical element 12 can be formed with different shapes without departing from the spirit and scope of the present invention. For example, a U-shaped portion could be used in place of the V-shaped portion to obtain substantially similar results.

The cylindrical element 12 of this stent 10 includes the double-curved portion (W) 21 located in the region of the valley where each interconnecting member 13 is connected to an adjacent cylindrical element 12. Peak portions (inverted V) 21 are adjacent to each double-curved portion (W) 20. Another valley portion (V) 23 connects each peak portion (inverted V) 21 to form the composite cylindrical ring. In this particular stent design, each of the strut junctions 24 (also referred to as “keyholes”) of each of the peak portions (inverted V) 22 and valley portions (V) 23 are V-shaped in order to provide closer nesting to provide a low-crimp profile. Although this particular stent pattern includes three interconnecting members connecting adjacent cylindrical elements 12 together, it is possible to use more or less interconnecting members. Generally, less interconnecting members result in a more flexible stent since the primary flexibility in the stent results from the cylindrical elements and especially the unsupported and unconstrained V-shaped elements. Of course, it is still possible to add interconnecting members if needed, to increase vessel scaffolding.

Referring again to FIGS. 4 and 5, the stent design of the present invention incorporates the use of varying lengths for the W- and V-shaped portions in order to provide additional nesting which allows the stent to be crimped to a low crimp profile. As can be best seen in FIGS. 5 and 5A, the length of the double-curved portion (W) 21 is substantially shorter than the adjacent valley portion (V) 23 which allows these elements to be positioned closer to each other during stent crimping. The arrows shown in FIG. 5 show the difference in the lengths of these particular portions which allows each valley portion (V) 23 to be crimped closer to the double-curved portion (W) 21 as is shown in FIG. 5A. This ability of the V-shaped portion to move closer to the W-shaped portion, referred to herein as “nesting”, is advantageous since a lower crimp profile can be achieved. While the term “nesting” may have different meanings in other patents and references, this term is used herein to describe the arrangement of elements described above and depicted in FIG. 5A. If these elements were the same length the edges of the strut junction 24 of the valley portion (V) 23 would strike the side of the double-curved portion (W) 21 much sooner preventing nesting of these elements. Thus, the stent design of the present invention provides a lower crimp profile.

Referring now to FIGS. 6 and 7, another embodiment of the present invention is shown. In this particular embodiment, the stent 25 includes cylindrical elements 12 which have both peak and valley portions generally having V, W and inverted V and inverted W shapes, in a repeating pattern to form each cylindrical element 12. Again, this serpentine pattern of the cylindrical element 12 has peaks and valleys which aid in the even distribution of expansion forces. Moreover, the stent 30 includes four interconnecting members 13 which connect adjacent cylindrical elements 12 together to form the composite stent.

The cylindrical elements of the stent include a double-curved portion (W) 21 located in the region of a valley where each interconnecting member 13 is connected to an adjacent cylindrical element 12. Peak portions (inverted V) 22 are located adjacent to the double-curved portion (W) 21. A valley portion (V) 23 is adjacent to each of the peak portions (inverted V) 22. Another peak portion (inverted W) 26 lies adjacent to each double-curved portion (W) 21. An interconnecting member 13 is also connected to the peak portion (inverted W) 26 for connecting the cylindrical element 12 to an adjacent cylindrical element. In this arrangement, interconnecting members 13 connect both peak portions of one cylindrical element to peak portions of an adjacent cylindrical element and valley portions of the same cylindrical element with a valley portion of an adjacent cylindrical element to provide additional scaffolding to the stent 30 to reduce the unsupported surface area in the body lumen.

As can be seen best in FIG. 7, the lengths of the double-curved portion (W) 21 and peak portion (inverted W) 26 are less than the adjacent peak portion (inverted V) 22 and valley portion (V) 23 to allow these elements of the cylindrical ring to nest and crimp to a smaller crimp profile. The differences in the lengths of these various elements are shown by the arrows in FIG. 7. This arrangement allows the V-shaped portion and W-shaped and inverted W-shaped portions to nest as is shown in FIG. 5A and described above.

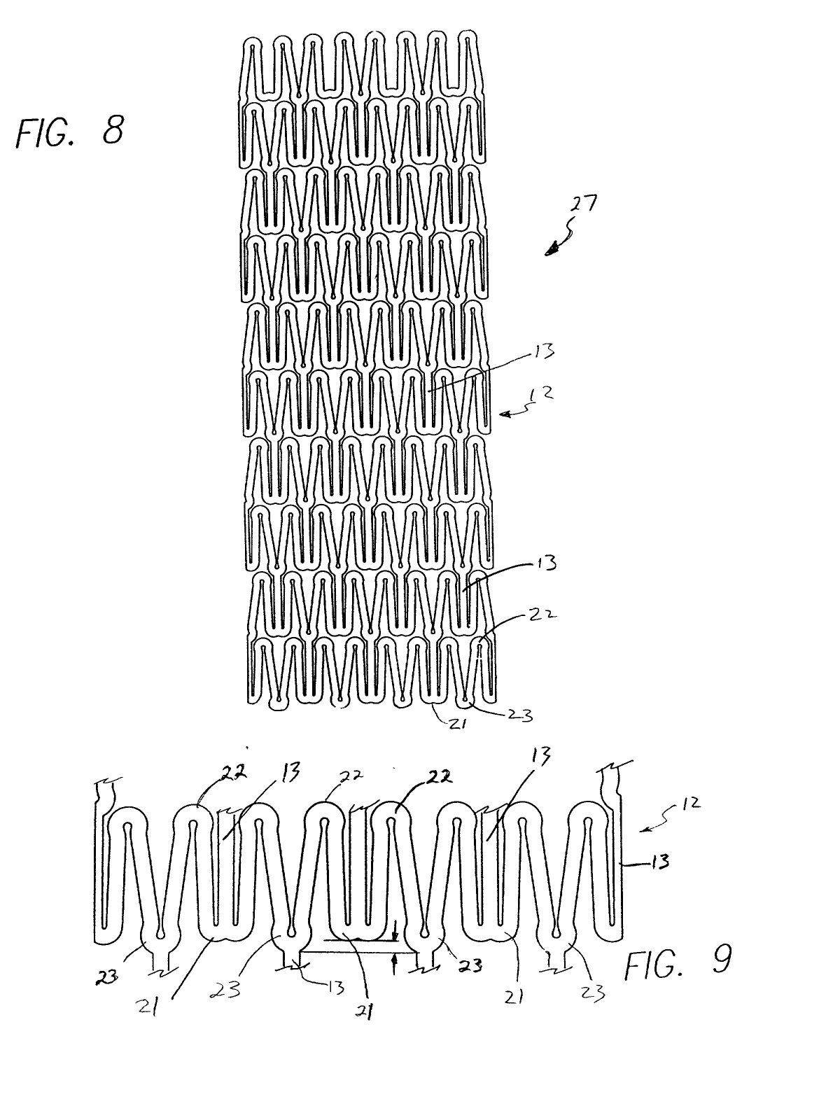

Referring now to FIGS. 8 and 9, another embodiment of a stent 27 is shown which includes cylindrical elements 12 having eight peaks and eight valleys per ring. In this particular design, the stent 27 includes cylindrical elements having strut arms which are shorter than the strut arms shown in the designs depicted in FIGS. 4-7 While shorter strut arms are being used, the circumference of the stent can remain the same since there are more V- and W-shaped portions forming the cylindrical ring. Additionally, due to the use of shorter strut arms, more rings can be utilized for the same stent length as those shown in the designs of FIGS. 4-7. As a result, there are more V- and W-shaped portions which provide greater coverage in the body lumen, thus helping to prevent tissue prolapse. Additionally, the struts forming the cylindrical elements of this design can be slightly thinner to accommodate a low-crimp size.

Still referring to FIGS. 8 and 9, each cylindrical element 12 includes four double-curved portions (W) 21 which sit adjacent to peak portions (inverted V) 22. Additional valley portions (V) 23 are found between each of the peak portions (inverted V) 22. Four interconnecting members 13 connect each of the double-curved portions (W) 21 with a valley portion (V) 23 of an adjacent cylindrical ring. Again, as can be seen best in FIG. 9, the length of the double-curved curved portion (W) 21 is shorter than the length of the adjacent valley portion (V) 23 to allow nesting which produces a low-crimp profile. Again, the novel use of shorter rings and additional spines should reduce the amount of unsupported surface area in the body lumen.

In many of the drawing figures, the present invention stent is depicted flat, in a plan view for ease of illustration. All of the embodiments depicted herein are cylindrically-shaped stents that are generally formed from tubing by laser cutting as described below.

One important feature of all of the embodiments of the present invention is the capability of the stents to expand from a low-profile diameter to a larger diameter, while still maintaining structural integrity in the expanded state and remaining highly flexible. Stents of the present invention each have an overall expansion ratio of about 1.0 up to about 4.0 times the original diameter, or more, from the as-cut diameter using certain compositions of stainless steel and crimped to approximately 75% of the as-cut diameter. For example, a 316L stainless steel stent of the invention can be radially crimped from a diameter of 1.0 unit down to a diameter of about 0.75 unit then expanded up to a diameter of about 4.0 units, which deforms the structural members beyond the elastic limit. The stents still retain structural integrity in the expanded state and will serve to hold open the vessel in which they are implanted. Materials other than stainless steel (316L) may afford higher or lower expansion ratios without sacrificing structural integrity.

The stents of the present invention can be made in many ways. However, one preferred method of making the stent is to cut a thin-walled tubular member, such as stainless steel tubing to remove portions of the tubing in the desired pattern for the stent, leaving relatively untouched the portions of the metallic tubing which are to form the stent. It may be preferred to cut the tubing in the desired pattern by means of a machine-controlled laser.

The tubing may be made of suitable biocompatible material such as stainless steel, nickel-titanium, or others. The stainless steel tube may be alloy-type:316L SS, Special Chemistry per ASTM F138-92 or ASTM F139-92 grade 2. Special Chemistry of type 3 16L per ASTM F138-92 or ASTM F139-92 Stainless Steel for Surgical Implants in weight percent.

| Carbon (C) | 0.03% | max. |
|---|---|---|
| Manganese (Mn) | 2.00% | max. |
| Phosphorous (P) | .025% | max. |
| Sulphur (S) | 0.010% | max. |
| Silicon (Si) | 0.75% | max. |
| Chromium (Cr) | 17.00-19.00% | |
| Nickel (Ni) | 13.00-15.50% | |
| Molybdenum (Mo) | 2.00-3.00% | |
| Nitrogen (N) | 0.10% | max. |
| Copper (Cu) | 0.50% | max. |

| | |
|---|---|
| Iron (Fe) | Balance |

The stent diameters are usually small, so the tubing from which it is made must necessarily also have a small diameter. For PTCA applications, typically the stent has an outer diameter on the order of about 1 mm (0.04 - 0.09 inches) in the unexpanded condition, the same outer diameter of the hypotubing from which it is made can be crimped to an outer diameter of about 0.06 inches, then can be expanded to an outer diameter of 8.0 mm or more. The wall thickness of the tubing is about 0.15 mm (0.005-0.010 inches). For stents implanted in other body lumens, such as PTA applications, the dimensions of the tubing are correspondingly larger. While it is preferred that the stents be made from laser cut tubing, those skilled in the art will realize that the stent can be laser cut from a flat sheet and then rolled up in a cylindrical configuration with the longitudinal edges welded to form a cylindrical member.

Generally, the tubing is put in a rotatable collet fixture of a machine-controlled apparatus for positioning the tubing relative to a laser. According to machine-encoded instructions, the tubing is then rotated and moved longitudinally relative to the laser which is also machine-controlled. The laser selectively removes the material from the tubing by ablation and a pattern is cut into the tube. The tube is therefore cut into the discrete pattern of the finished stent. Further details on how the tubing can be cut by a laser are found in U.S. Pat. Nos. 5,759,192 (Saunders) and 5,780,807 (Saunders), which have been assigned to Advanced Cardiovascular Systems, Inc.

The process of cutting a pattern for the stent into the tubing generally is automated except for loading and unloading the length of tubing. For example, a pattern can be cut in tubing using a CNC-opposing collet fixture for axial rotation of the length of tubing, in conjunction with CNC X/Y table to move the length of tubing axially relative to a machine-controlled laser as described. The entire space between collets can be patterned using the CO₂ or Nd:YAG laser set-up. The program for control of the apparatus is dependent on the particular configuration used and the pattern to be ablated in the coding.

It should be appreciated that the stent assembly can be made from either pseudo elastic stress-induced martensite NiTi or shape-memory NiTi. A suitable composition of Nitinol used in the manufacture of a self expanding stent of the present invention is approximately 55% nickel and 45% titanium (by weight) with trace amounts of other elements making up about 0.5% of the composition. The austenite transformation temperature is between about −15° C. and 0° C. in order to achieve superelasticity. The austenite temperature is measured by the bend and free recovery tangent method. The upper plateau strength is about a minimum of 60,000 psi with an ultimate tensile strength of a minimum of about 155,000 psi. The permanent set (after applying 8% strain and unloading), is approximately 0.5%. The breaking elongation is a minimum of 10%. It should be appreciated that other compositions of Nitinol can be utilized, as can other self-expanding alloys, to obtain the same features of a self-expanding stent made in accordance with the present invention.

One way of making the stent of the present invention is to utilize a shape-memory material, such a nickel titanium, which has the struts cut using a machine-controlled laser. A tubular piece of material could be utilized in this process. The struts of the stent could be manufactured to remain in its open position while at body temperature and would move to its collapsed position upon application of a low temperature. One suitable method to allow the stent to assume a phase change would facilitate the stent being mounted onto the delivery catheter and chilled in a cooling chamber maintained at a temperature below the martensite finish temperature through the use of liquid nitrogen, for example. Once the stent assumes its collapsed position, the restraining sheath can be placed over the stent to prevent the device from expanding once the temperature is brought up to body temperature. Thereafter, once the stent is to be utilized, the restraining sheath is simply retracted to allow the stent to move to its expanded position within the patient's vasculature.

The stent can be electro polished to obtain a smooth finish with a thin layer of titanium oxide or other suitable material placed on the surface. The stent is usually implanted into the target vessel which is smaller than the stent diameter so that the stent applies a force to the vessel wall to keep it open.

The stent tubing of a self expanding stent made in accordance with the present invention may be made of suitable biocompatible material besides super-elastic nickel-titanium (NiTi) alloys. In this case the stent would be formed full size but deformed (e.g. compressed) to a smaller diameter onto the balloon of the delivery catheter to facilitate intra lumenal delivery to a desired intra lumenal site. The stress induced by the deformation transforms the stent from an austenite phase to a martensite phase, and upon release of the force when the stent reaches the desired intra lumenal location, allows the stent to expand due to the transformation back to the more stable austenite phase.

While the invention has been illustrated and described herein in terms of its use as intra vascular stents, it will be apparent to those skilled in the art that the stents can be used in other instances in all conduits in the body, such as, but not limited to, the urethra and esophagus. Other modifications and improvements may be made without departing from the scope of the invention.

What is claimed is:

1. A stent for implanting in a body lumen, comprising:
a plurality of adjacent cylindrical elements each having a circumference extending about a longitudinal stent axis and being substantially independently expandable in a radial direction, each cylindrical element being arranged in alignment along the longitudinal stent axis and formed in a generally serpentine wave pattern transverse to the longitudinal axis and containing alternating valley portions and peak portions, wherein at least two adjacent valley portions or two adjacent peak portions on each cylindrical element is capable of nesting when the stent is crimped or collapsed; and
a plurality of interconnecting members extending between the adjacent cylindrical elements and connecting adjacent cylindrical elements to one another.

2. The stent of claim 1, wherein:
at least two valley portions in each cylindrical element have differing longitudinal lengths which permits nesting of the cylindrical element.

3. The stent of claim 2, wherein:
one valley portion is a V-shaped portion and the other adjacent valley portion is a W-shaped portion having different longitudinal lengths.

4. The stent of claim 3, wherein:
the W-shaped valley portion is smaller in length than the V-shaped valley portion.

5. The stent of claim 1, wherein:
at least two adjacent peak portions in each cylindrical element have differing longitudinal lengths which permits nesting of the cylindrical element.

6. The stent of claim 5, wherein:
one peak portion is a V-shaped portion and the adjacent peak portion is a W-shaped portion having different longitudinal lengths.

7. The stent of claim 6, wherein:
the W-shaped peak portion has a longitudinal length less than the V-shaped peak portion.

8. The stent of claim 1, wherein:
at least two adjacent peak portions in each cylindrical element have differing longitudinal lengths which permit nesting and at least two adjacent valley portions in each cylindrical element have differing longitudinal lengths which permits nesting of the valley portions.

9. The stent of claim 8, wherein:
one peak portion is a V-shaped portion and an adjacent peak portion is a W-shaped portion and one valley portion is a V-shaped portion and an adjacent valley portion is a W-shaped portion.

10. The stent of claim 9, wherein:
the W-shaped portion of both the valley portion and peak portion has a longitudinal length smaller than the V-shaped portion of the peak portion and valley portion.

11. The stent of claim 1, wherein:
the interconnecting members connect W-shaped valley portions with V-shaped valley portions of adjacent cylindrical elements.

12. The stent of claim 10, wherein:
the interconnecting members connect W-shaped valley portions and W-shaped peak portions with each adjacent cylindrical element.

13. The stent of claim 12, wherein:
the interconnecting member connects W-shaped valley portions with V-shaped valley portions on adjacent cylindrical elements.

14. The stent of claim 13, wherein:
each cylindrical element has a plurality of valley portions having a W-shape and wherein adjacent cylindrical elements are arranged so that the W-shaped valley portions are out of phase.

15. The stent of claim 12, wherein:
each cylindrical element has at least two peak portions having a W-shaped portion and two valley portions having a W-shaped portion.

16. The stent of claim 15, wherein:
the W-shaped peak portion and W-shaped valley portion are arranged adjacent to each other on each cylindrical element.

17. The stent of claim 1, wherein:
each cylindrical element includes at least four valley portions having a W-shaped portion.

18. The stent of claim 17, wherein:
each cylindrical element has four valley portions having a V-shape which are adjacent to each of the W-shaped valley portions.

19. The stent of claim 18, wherein:
the W-shaped valley portions on each cylindrical element has a longitudinal length which is less than the longitudinal length of an adjacent V-shaped valley portion.

20. The stent of claim 19, wherein:
each cylindrical element has eight peak portions and eight valley portions.

21. The stent of claim 1, wherein:
the stent is expandable from a collapsed position to an expanded position by the application of a controlled external force.

22. The stent of claim 1, wherein:
the stent is made from a self-expanding material which allows the stent to move between a collapsed position and an expanded position.

23. A stent for implanting in a body lumen, comprising:
a plurality of adjacent cylindrical elements each having a circumference extending about a longitudinal stent axis and being substantially independently expandable in a radial direction, each cylindrical element being arranged in alignment along the longitudinal stent axis and formed in a generally serpentine wave pattern transverse to the longitudinal axis and containing alternating valley portions and peak portions, wherein at least two adjacent valley portions or two adjacent peak portions on each cylindrical element is capable of nesting when the stent is crimped or collapsed; and
means for connecting adjacent cylindrical elements together.
